(19) United States
(12) Patent Application Publication
(10) Pub. No.: US 2010/0160667 A1
(43) Pub. Date: Jun. 24, 2010

(54) Shaped Catalyst Bodies with Characteristics of Ion Exchangers

(75) Inventors: Marco Bosch, Antwerpen (BE); Nawid Kashani-Shirazi, Liveshime (DE); Christian Kuhrs, Heidelberg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/376,975

(22) PCT Filed: Aug. 7, 2007
(86) PCT No.: PCT/EP2007/058201

§ 371 Date: Feb. 10, 2009

(30) Foreign Application Priority Data
Aug. 10, 2006 (EP) 06118733.2

(51) Int. Cl. C07C 69/02; B01J 37/30; B01J 29/18; B01J 29/08

(52) U.S. Cl. 560/129; 502/11; 502/78; 502/79

(57) ABSTRACT

Processes comprising: (a) providing a shaped body having a surface and comprising a metal oxide, and impregnating the shaped body with a liquid comprising an unsaturated compound; (b) subjecting the impregnated shaped body to thermolysis under an inert gas atmosphere at a temperature of 250 to 400° C., such that at least 10% by weight of the unsaturated compound is thermalized to form a layer of a high molecular weight polyaromatic on at least a portion of the surface of the shaped body; and (c) reacting the shaped body with a reagent to functionalize the polyaromatic layer, wherein the shaped body has a smallest dimension in any spatial direction of at least 1 mm to provide a shaped catalyst body having ion- exchange properties; shaped catalyst bodies prepared thereby and uses therefor as catalysts.

The invention relates to shaped catalyst bodies having ion-exchange properties, a process for producing shaped catalyst bodies having ion-exchange properties and the use of shaped catalyst bodies having ion-exchange properties for chemical reactions.

Ion exchangers are water-insoluble but hydratable solids which are able to bind ions from a solution and at the same time release other ions having a charge of the same sign to the solution, with the balance between the total charge adsorbed and the total charge released being zero. Depending on the charge on the exchanged ions, the ion exchangers are referred to as cation or anion exchangers. Both inorganic ion exchangers which are in part of natural origin, e.g. zeolites, montmorillonites, bentonites, attapulgites and other aluminosilicates (cf. A.F. Holleman, E. Wiberg, Lehrbuch der Anorganischen Chemie, 91st-100th edition, 1985, W. de Gruyter, Berlin, on pages 771 to 778), and organic ion exchangers based on polymers are known.

Organic ion exchangers comprise a high molecular weight polymer matrix to which ionic groups are bound. Known examples are sulfonated polystyrenes and polystyrene- divinylbenzene copolymers, polyacrylates, phenol-formaldehyde resins and polyalkylamine resins (cf. Ullmann's Encyclopedia of Industrial Chemistry, 6th Edition, 2000 Electronic Release, chapter “Ion Exchangers”).

The main application area for ion exchangers is water treatment. In addition, anionic ion exchangers, for example, are employed as acidic heterogeneous catalysts in chemical reactions. Typical acid-catalyzed reactions are the esterification of carboxylic acids by means of alcohols, the etherification of alcohols, the addition of water onto olefins, the dimerization and oligomerization of olefins, the addition of olefins onto aromatics, the acylation of aromatics and hydrolyses.

Reactions of carboxylic acids with alcohols to form esters over ion exchangers in a reactive distillation are of particular interest. The removal of the water of reaction by this method enables the yield of esters to be increased to above the equilibrium distribution.

When ion exchangers based on polystyrene are employed in a reactive distillation, they are usually used in the form of relatively small particles whose largest dimension is typically in the range from 0.2 to 1.0 mm. It has been found that larger ion exchanger particles are not stable under reaction conditions. They swell, resulting in the polymer matrix being destroyed and the particle disintegrating. Accordingly, organic ion exchangers can only be used for a reactive distillation when they are installed in a permeable container, e.g. a mesh bag. Such bags can, for example, comprise a woven wire mesh and either serve directly as distillation internals (e.g. KATAPAK® S from Sulzer AG, CH-8404 Winterthur) or be inserted as flat bags between the individual layers of the distillation packing (e.g. Multipak® from Montz GmbH, D-40723 Hilden, or “bales” from CDTech, Houston, USA). However, the use of these packings is susceptible to malfunctions and has the disadvantage that in processes in which the catalyst is surrounded by a gas/liquid mixture, the matching irrigation density has to be adhered to precisely, which proves to be difficult in practice. In addition, the wetting of the catalyst particles is not ideal so that only part of the catalyst material present in the bag actually participates in the reaction. Furthermore, replacement of the catalyst is very complicated.

There is therefore a need for catalysts having ion-exchange properties in the form of relatively large particles whose size and shape correspond to the shaped catalyst bodies which are otherwise customary, i.e., for example, rings, extrudates, cylinders or tubes having dimensions in the range from a few millimeters to a few centimeters. Such shaped bodies can be employed in a simple fashion without mesh bags or similar devices by pouring them onto the column trays, so that replacement of the catalyst is readily possible.

Some studies on the production of such shaped catalyst bodies having ion-exchange properties are known from the prior art. For example, DE 1285170 describes a process for producing catalytically active shaped bodies comprising ion-exchange resins in which the ion exchanger is embedded in a thermoplastic polymer as matrix. However, the cylinders having a size of from 1 to 5 cm which are obtainable by this process have only a low stability.

U. Kunz, U. Hoffmann, Preparation of Catalysts VI (G. Poncelet et al., Eds.), Elsevier 1995, pp. 299-308, describe the loading of commercially available catalyst supports (including ceramic Raschig rings, glass or silicon carbide foams having a size of from about 1 to 2 cm) with an ion exchanger. Here, the polymer matrix is firstly deposited on the supports by means of a free-radically initiated precipitation polymerization in the presence of a pore former, the pore former is subsequently extracted and the material is finally activated by sulfonation. However, the method of production is problematical, since the polymerization takes place not only on the surface of the support. In addition, the catalytic activity of the products was low compared to commercially available ion exchangers.

It was an object of the present invention to provide catalysts having ion-exchange properties in the form of relatively large particles. A further object of the present invention was to develop a process for producing the inventive shaped catalyst bodies having ion-exchange properties and methods of using the inventive shaped catalyst bodies having ion-exchange properties for chemical reactions.

According to the invention, this object is achieved by a process for producing shaped catalyst bodies having ion-exchange properties, which comprises the steps
a) impregnation of shaped bodies comprising at least one metal oxide with a liquid comprising at least one unsaturated compound,
b) thermolysis of at least 10% by weight of the unsaturated compound comprised in the shaped bodies after step a) to form a high molecular weight polymer layer, and
c) reaction of the shaped bodies obtained after step b) with a sulfonating reagent to sulfonate the polymer layer,

where the smallest dimension of the shaped bodies in any spatial direction is at least 1 mm.

According to the invention, the shaped bodies comprise at least one metal oxide. This can be an oxide of magnesium, aluminum, silicon, titanium, chromium, iron, gallium, germanium, zirconium, niobium, tin, lanthanum or praseodymium and also mixtures thereof. The metal oxide content of the shaped bodies is generally in the range from 3 to 100% by weight, preferably from 10 to 99% by weight.

Shaped bodies comprising aluminum oxide or silicon oxide or aluminosilicates are particularly useful. Preference is given to aluminosilicates having a zeolite structure of the mordenite, LTA, LTL, FAU, BEA, KFI, FER, DDR, MFI or MEL type which are at least partly present in the H⁺ and/or NH₄⁺ form (see Atlas of Zeolite Framework Types, Ch. Baerlocher, W. M. Meier, D. H. Olson, 5th Revised Edition, 2001, Elsevier).

Particular preference is given to shaped bodies which comprise an aluminosilicate and also an oxide of silicon, of aluminum, of titanium, of zirconium or mixtures thereof. In such cases, the aluminosilicate content of the shaped bodies is preferably in the range from 5 to 95% by weight, based on the sum of all metal oxides.

Very particularly suitable shaped bodies are shaped bodies whose composition corresponds to that of FCC catalysts (FCC=fluid catalytic cracking). The FCC catalysts are known per se to those skilled in the art and generally comprise a Y-zeolite (a low-aluminum form of faujasite) which is embedded together with fillers, binders and additives in an active matrix.

The shaped bodies to be used according to the invention generally have a specific surface area (measured by the BET method of Brunauer-Emmet-Teller) of from 50 to 1200 m²/g, preferably from 100 to 800 m²/g and particularly preferably from 200 to 700 m²/g.

The shaped bodies to be used according to the invention can have any three-dimensional shape, but their smallest dimension in any spatial direction is at least 1 mm, preferably at least 5 mm. Such shaped bodies cannot pass through a sieve plate having a mesh opening of 1 mm (or preferably 5 mm). The comparatively large dimensions of the shaped bodies to be used according to the invention makes them simple to use as a bed on meshes or perforated plates, for example in reactors or distillation columns, so that the internals or fastening means such as wire or mesh bags required for finely divided materials are not necessary and replacement of the shaped bodies is readily possible. Examples of suitable shaped bodies are Raschig rings, extrudates, cylinders, pellets, crosses, granules, compacts, tubes or other structures which are in part commercially available or can be produced by methods with which those skilled in the art are familiar. The choice of the most favorable shaped body for the particular application should be made taking fluid-dynamic aspects and also the reactor size and reactor type into account.

According to the process of the invention, the shaped bodies are impregnated with a liquid. For the present purposes, impregnation is the mixing of the shaped bodies with a liquid which is taken up to a certain extent by the shaped bodies. If the amount of liquid used exceeds the uptake capacity of the shaped bodies, a supernatant liquid is present above the shaped bodies and has to be separated off from the shaped bodies, for example by decantation or filtration, before they are used further. The impregnation is usually carried out at a temperature of from 0 to 75° C. under an inert gas atmosphere comprising, for example, nitrogen and/or argon, using an amount of from 0.01 g to 2.0 g of liquid per gram of shaped bodies.

According to the invention, the liquid comprises at least one unsaturated compound which reacts in the thermolysis step b) to form a high molecular weight polymer layer. Possible unsaturated compounds are monomers in general which are liquid or soluble under the impregnation conditions and have at least one carbon-carbon double and/or triple bond and are capable of undergoing a polymerization reaction, for example styrene, divinyibenzene, acrylic acid, methacrylic acid, C₁-C₁₂-alkyl acrylates, C₁-C₁₂-alkyl methacrylates, acrylamide, methacrylamide, acrylonitrile, N-vinyl-C₁-C₁₂-carboxamides, maleic acid, fumaric acid, crotonic acid, allylacetic acid, N-vinyl-pyrrolidone, C₂-C₁₂-olefins or C₂-C₁₂-alkynes. Preferred unsaturated compounds are styrene, divinylbenzene, acrylic acid, methacrylic acid, C₁-C₁₂-alkyl acrylates, C₁-C₁₂-alkyl methacrylates, acrylamide and acrylonitrile.

The impregnation of shaped bodies in step a) of the process of the invention is particularly preferably carried out using a liquid comprising styrene. The styrene-comprising liquid can be pure styrene but can also comprise further unsaturated compounds, for example divinylbenzene, acrylates, C₁-C₁₂-alkyl methacrylates, acrylamide, methacrylamide and acrylonitrile. The proportion of styrene in the impregnation liquid is preferably at least 50% by weight based on the total amount of monomers. Very particular preference is given to an impregnation liquid which comprises from about 85 to 100% by weight of styrene and from 15 to 0% by weight of divinylbenzene, in each case based on the total amount of monomers.

In addition, the impregnation liquid can further comprise organic solvents which are miscible with the unsaturated compounds, for example hydrocarbons such as benzene, toluene, cyclohexane or pentane or ethers such as tetrahydrofuran, diethyl ether or dioxane. The concentration of unsaturated compounds in a solvent-comprising impregnation liquid is usually at least 30% by weight, preferably at least 50% by weight and particularly preferably at least 80% by weight.

In the process of the invention, the impregnated shaped bodies obtained after step a) are subsequently thermalized in step b). Here, a (co)polymerization of the unsaturated compounds taken up by the shaped bodies induced thermally and/or by interaction with the material of the shaped bodies takes place (cf. US 3352800). According to the invention, the thermolysis is carried out in an inert gas atmosphere at temperatures above 75° C. in such a way that at least 10% of the unsaturated compounds taken up by the shaped bodies (co)polymerize. The thermolysis is preferably carried out under a nitrogen atmosphere at temperatures of from 250 to 400° C., with the shaped bodies being maintained in this temperature range for at least 30 minutes. The thermolysis is particularly preferably carried out in an autoclave at a temperature of from 300 to 400° C. and a pressure of from 5 to 100 bar for a time of from 2 to 10 hours. During this treatment, not only does complete polymerization of the unsaturated compounds taken up by the shaped bodies take place, but further condensation of the polymer or copolymer formed takes place to form a high molecular weight layer of polyaromatics on the surface of the shaped bodies, which become brown or black in color as a result.

The specific surface area of the thermolysed shaped bodies (determined by the BET method) is usually in the range from 30 to 500 m²/g, preferably in the range from 50 to 300 m²/g.

The shaped bodies can optionally be washed with an organic solvent after the thermolysis step b) in order to remove excess polymer or residual monomers. Alcohols, e.g. methanol, ethanol, isopropanol or n-butanol, are preferably used for this purpose.

The shaped bodies obtained after step b) of the process of the invention are finally functionalized in step c). Here, the high molecular weight layer of polyaromatics on the surface of the shaped bodies is reacted with at least one reagent to provide it with functional groups which are able to exchange ions with the surroundings.

In order to obtain an anionic exchanger, the high molecular weight layer of polyaromatics can, for example, be sulfonated. The sulfonation is typically effected by reaction of the shaped bodies with concentrated or dilute sulfuric acid at temperatures of from 25° C. to 100° C., by reaction with chlorosulfonic acid in the liquid phase in the presence of acetone, chloroalkanes such as methylene chloride, chloroform or dichlorethanes and/or acetonitrile at temperatures of from 20 to 90° C., by reaction with amidosulfuric acid in water at temperatures of from 60 to 100° C. or by reaction with gaseous sulfur trioxide at temperatures of from 50 to 120° C. The shaped catalyst bodies obtainable by these methods have an acid density of at least 0.1 mmol/g and cutting hardness of at least 1 N.

Cationic ion exchangers can be obtained, for example, by reaction of the shaped bodies with sulfuryl chloride and subsequent reaction of the product with a tertiary amine, e.g. trimethylamine, dimethylethylamine, triethylamine, tripropylamine or tributylamine, to form quaternary ammonium groups.

After the functionalization step c), the shaped bodies may be washed again if appropriate. Particularly after a sulfonylation, washing with alcohols, water and/or dilute sulfuric acid at temperatures of from 10 to 80° C. is recommended in order to remove any excess sulfonating reagent present.

The functionalized shaped bodies preferably have a specific surface area (determined by the BET method) of from 10 to 250 m²/g, particularly preferably from 30 to 200 m²/g.

The shaped catalyst bodies according to the invention can be used for all chemical processes which can be catalyzed by ion exchangers. The reactions which can be catalyzed by the shaped catalyst bodies according to the invention comprise, for example, acetalizations, eliminations of water and hydrogen halides, adduct formation and addition reaction, hydrolyses, esterifications and transesterifications, condensations, epoxidations, rearrangements, polymerizations and acylations.

The shaped catalyst bodies according to the invention can be used particularly advantageously in reactive distillations during which a reaction product is removed from the catalyst bed in the amount in which it is formed. When the reaction is, for example, carried out in a column, it is advantageous to introduce the high-boiling starting materials above the reaction zone comprising the catalyst bed and introduce the low- boiling starting materials below this reaction zone and take off the reaction products formed from the column at suitable places determined by their boiling points. in particular, the shaped catalyst bodies according to the invention are also suitable for use in multichannel packings as are described, for example, in EP 1614462. Owing to their dimensions and their shape, the shaped catalyst bodies according to the invention can easily be introduced into and removed again from such multichannel packings by simply pouring them onto the multichannel packing and allowing them to trickle into or through the channels provided.

The following examples illustrate the invention but do not restrict its scope.

### EXAMPLE 1

2 mm extrudates as shaped bodies were produced by kneading 1800 g of a beta-zeolite (TZB 213 from Tricat Zeolites GmbH, D-06749 Bitterfeld), 1125 g of Ludox® AS 40 (colloidal silica gel, 40% strength suspension in water, CAS No. 7631-86-9, from Aldrich), 112.5 g Walocel® (from Wolff Waisrode AG, obtainable via PUFAS Werke KG/decotric GmbH, D-34334 Hannoversch Munden) and 2280 ml of deionized water in a kneader for 4 hours and subsequently extruding the mixture at 140 bar. The extrudates were then dried at 120° C. for 16 hours in a convection drying oven and calcined at 500° C. for 5 hours in a muffle furnace (heating rate: 2° C./min). Yield: 1911.8 g having a cutting hardness of 4.9 N.

500 g of these extrudates were placed together with 125 ml of styrene (>99.5%, from Fluka) in a glass vessel and mixed well (no color change). The impregnated extrudates were then introduced into an autoclave, the autoclave was flushed with nitrogen and subsequently heated at 380° C. for 5 hours, resulting a pressure of about 58-61 bar being established. After cooling, the extrudates had become black.

270 g of these black extrudates were subsequently placed in an exchanger tube which is located on a 1 l four-necked flask provided with nitrogen attachment, dropping funnel, thermometer and glass pressure release valve (0.2 bar) with three downstream wash bottles (first bottle empty, 2nd and 3rd filled with 5% strength sodium hydroxide solution) and is heated to 80° C. together with the flask. A stream of nitrogen of 301/h was passed from the bottom upward through the exchange tube (offgas passed directly to exhaust) and 200 ml of oleum having a free sulfur trioxide content of 65% by weight was then introduced dropwise into the flask over a period of 1 hour. The heating was then switched off and the flask was flushed with nitrogen for a further 30 minutes. The extrudates were removed, washed with deionized water until the filtrate was colorless and then dried at 160° C. for 5 hours in a convection drying oven.

Yield: 336 g

Analysis: S: 5.6%

### EXAMPLE 2

60 g of aluminum oxide extrudates (>99% of Al₂O₃) were placed together with 15 ml of styrene (>99.5%, from Fluke) in a glass vessel and mixed well (no color change). The impregnated extrudates were then introduced into an autoclave, the autoclave was flushed with nitrogen and subsequently heated at 380° C. for 5 hours, resulting a pressure of about 9-10 bar being established. After cooling, the extrudates had become brown.

15 g of these brown extrudates were subsequently placed together with 20 ml of acetone (>99.9% strength) and 2.7 g of chlorosulfonic acid (99% strength, from Aldrich) in a round-bottomed flask provided with reflux condenser and heated at about 70° C. for 1 hour (reflux). The extrudates were subsequently filtered off and washed with acetone until the washings no longer fumed (using about 1 l of acetone). The extrudates were then washed with about 2.5 l of deionized water/methanol (>99.8% strength, from Fluke) (60:40 v/v) and then dried at 120° C. and 100 mbar for 16 hours.

Yield: 11.16 g (light-brown extrudates)

Analysis: S: 4.6%

### EXAMPLE 3

2 mm extrudates were produced, treated with styrene and thermolyzed in an autoclave in a manner analogous to the description in example 1.

10 g of the black extrudates obtained were subsequently placed together with 20 ml of chloroform and 10 g of sulfuric acid (95-97% strength) in a round-bottomed flask provided with reflux condenser and refluxed for 1 hour. After cooling, the extrudates were transferred to a suction filter and washed firstly with about 1 l of chloroform and then with about 2 l of deionized water, The extrudates were finally dried at 160° C. and 100 mbar for 4 hours.

Analysis: S: 4.5%

### EXAMPLE 4

2 mm extrudates were produced, treated with styrene and thermolyzed in an autoclave in a manner analogous to the description in example 1.

10 g of the black extrudates obtained were subsequently placed together with 10 g of amidosulfuric acid and 50 ml of deionized water in a round-bottomed flask provided with reflux condenser and refluxed for 1 hour. After cooling, the extrudates were transferred to a suction filter and washed with about 3 l of deionized water. The extrudates were subsequently dried at 160° C. and 100 mbar for 4 hours.

Analysis: S: 2.9%

### EXAMPLE 5

2 mm extrudates were produced as shaped bodies by kneading 200 g of an FCC catalyst of the NaphthaMax® type (from BASF Catalysts LLC) having a specific surface area of 200 m²/g and an average particle size of 75 μm with 50 g of Pural® SB (aluminum oxide, from Sasol Germany GmbH, Hamburg), 7.5 g of formic acid, 9 ml of nitric acid (65% strength) and 110 ml of deionized water in a kneader for 50 minutes and the mixture was subsequently extruded at 140 bar. The extrudates were then dried at 120° C. in a convection drying oven for 16 hours and calcined at 500° C. in a muffle furnace for 5 hours (heating rate: 2° C./min).

80 g of these white extrudates were placed together with 18 ml of styrene (>99.5%, from Fluke) in a glass vessel and mixed well (no color change). The impregnated extrudates were then introduced into an autoclave, the autoclave was flushed with nitrogen and subsequently heated at 380° C. for 5 hours. After cooling, the extrudates had become brown.

91 g of these brown extrudates were subsequently placed in a 500 ml round-bottom flask provided with a nitrogen attachment and three downstream wash bottles (first bottle empty, 2nd and 3rd bottles filled with 5% strength sodium hydroxide solution) and admixed with 150 ml of oleum having a free sulfur trioxide content of 65% by weight at room temperature. The offgases were taken off by suction. After 1 hour, water was gradually added until the acid was diluted. The liquid was decanted off from the extrudates and the extrudates were washed a number of times with deionized water and then dried at 120° C. under reduced pressure for 16 hours.

Yield: 110 g (black extrudates)

Analysis: S: 13.5%

### EXAMPLE 6

The shaped bodies produced in example 1 were installed in a tube reactor operated with circulation from a reservoir. Butanol and acetic acid with a slight molar excess of butanol (1.2:1.0) were introduced into the reservoir. The apparatus was heated to 100° C., resulting in the liquid present therein being heated to about 90° C. Downstream of the reactor, the mixture was depressurized to atmospheric pressure via an overflow valve and cooled back down to room temperature.

In a second identical experimental set-up, Amberlyst® 48 from Rohm+Haas was used as catalyst in place of the shaped bodies produced in example 1.

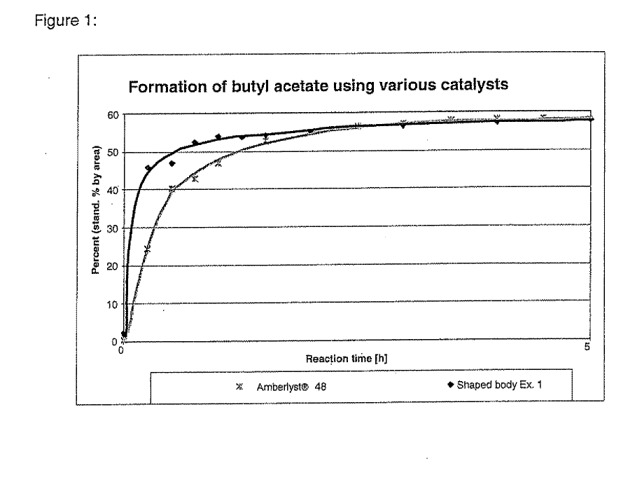

In both experiments, samples were taken at regular intervals and analyzed to determine their butyl acetate content. In FIG. 1, the concentration of butyl acetate formed (in GC-% by area) is plotted against the reaction time for both experiments.

1-16. (canceled)

17. A process comprising:
(a) providing a shaped body having a surface and comprising a metal oxide, and impregnating the shaped body with a liquid comprising an unsaturated compound;
(b) subjecting the impregnated shaped body to thermolysis under an inert gas atmosphere at a temperature of 250 to 400° C., such that at least 10% by weight of the unsaturated compound is thermalized to form a layer of a high molecular weight polyaromatic on at least a portion of the surface of the shaped body; and
(c) reacting the shaped body with a reagent to functionalize the polyaromatic layer,
wherein the shaped body has a smallest dimension in any spatial direction of at least 1 mm to provide a shaped catalyst body having ion-exchange properties.

18. The process according to claim 17, wherein the metal oxide comprises one or more selected from the group consisting of oxides of magnesium, aluminum, silicon, titanium, chromium, iron, gallium, germanium, zirconium, niobium, tin, lanthanum, praseodymium and mixtures thereof.

19. The process according to claim 17, wherein the metal oxide comprises an aluminosilicate having a zeolite structure selected from the group consisting of mordenite, LTA, LTL, FAU, BEA, KFI, FER, DDR, MFI and MEL, and wherein the aluminosilicate is at least partly present in a H⁺ and/or NH₄⁺ form.

20. The process according to claim 17, wherein the shaped body provided in (a) has a fluid catalytic cracking catalyst composition.

21. The process according to claim 19, wherein the shaped body provided in (a) has a fluid catalytic cracking catalyst composition.

22. The process according to claim 17, wherein the shaped body provided in (a) is impregnated with an amount of 0.01 to 2 grams of liquid per gram of the shaped body.

23. The process according to claim 19, wherein the shaped body provided in (a) is impregnated with an amount of 0.01 to 2 grams of liquid per gram of the shaped body.

24. The process according to claim 17, wherein the unsaturated compound comprises one or more selected from the group consisting of styrene, divinylbenzene, acrylic acid, methacrylic acid, C₁-C₁₂-alkyl acrylates, C₁-C₁₂-alkyl methacrylates, acrylamides, methacrylamides, acrylonitrile, N-vinyl-C₁-C₁₂-carboxamides, maleic acid, fumaric acid, crotonic acid, allylacetic acid, N-vinylpyiTolidone, C₂-C₁₂-olefins, C₂-C₁₂-alkynes and mixtures thereof.

25. The process according to claim 17, wherein the unsaturated compound comprises styrene.

26. The process according to claim 19, wherein the unsaturated compound comprises styrene.

27. The process according to claim 23, wherein the unsaturated compound comprises styrene.

28. The process according to claim 17, wherein the unsaturated compound comprises a mixture of styrene and one or more compounds selected from the group consisting of divinylbenzene, acrylic acid, methacrylic acid, C₁-C₁₂-alkyl acrylates, C₁-C₁₂-alkyl methacrylates, acrylamides, methacrylamides and acrylonitrile.

29. The process according to claim 17, wherein the reagent comprises a sulfonating reagent selected from the group consisting of sulfuric acid, chlorosulfonic acid, amidosulfuric acid, sulfur trioxide and mixtures thereof.

30. The process according to claim 17, wherein reacting the shaped body with a reagent comprises first reacting the shaped body with sulfuryl chloride and subsequently with a tertiary amine.

31. A shaped catalyst body having ion-exchange properties prepared by a process according to claim 17.

32. A shaped catalyst body having ion-exchange properties prepared by a process according to claim 19.

33. A shaped catalyst body having ion-exchange properties prepared by a process according to claim 23.

34. A shaped catalyst body having ion-exchange properties prepared by a process according to claim 27.

35. The shaped catalyst body according to claim 31, having an acid density of at least 0.1 mmol/g and a cutting hardness of at least 1 N,

36. A process comprising reacting one or more components in the presence of a catalyst comprising the shaped catalyst body according to claim 31.
